(19) United States
(12) Patent Application Publication
(10) Pub. No.: US 2019/0161717 A1
(43) Pub. Date: May 30, 2019

(54) VASCULAR TUBULAR HUMAN BLOOD BRAIN BARRIER DEVICE

(71) Applicant: StemoniX Inc., Eden Prairie, MN (US)

(72) Inventor: Robert John Petcavich, The Woodlands, TX (US)

(21) Appl. No.: 16/323,118

(22) PCT Filed: Aug. 2, 2017
(86) PCT No.: PCT/US2017/045114

§ 371 Date: Feb. 4, 2019

Related U.S. Application Data
(60) Provisional application No. 62/371,491, filed on Aug. 5, 2016.

(51) Int. Cl. C12M 1/12; C12M 1/00; C12Q 1/02

(52) U.S. Cl. CPC C12M 25/10; C12Q 1/025; C12M 23/20; C12M 23/06

(57) ABSTRACT

The present disclosure provides an in vitro tubular blood brain barrier mimic used to model drug transport across the brain capillary endothelial barrier cells to neurons. In one embodiment the stack is comprised of interior neuro endothelial capillary cells, extracellular matrix, porous polymeric hollow fiber, exterior extracellular matrix, and neuron astrocytes. The tubular human blood brain barrier mimic is used to test molecular transport and effects of drug candidates across the multilayer stack.

### CROSS REFERENCE TO RELATED APPLICATIONS

This application claims the benefit of the filing date of U.S. application Ser. No. 62/371,491, filed on Aug. 5, 2016, the disclosure of which is incorporated by reference herein.

### FIELD OF THE DISCLOSURE

This disclosure relates to a method of preparing a human vascular mimic tubular multilayer cellular stack blood brain barrier (BBB) device in vitro. The multilayer stack can emulate a human BBB and response to the effects of various therapeutic treatment challenges presented to brain endothelial capillary cell side layer of the BBB tubular human mimic may be monitored.

### BACKGROUND

The blood-brain barrier (BBB) is a highly selective permeability barrier that separates the circulating blood from the brain extracellular fluid in the central nervous system (CNS). The blood-brain barrier is formed by brain endothelial cells, which are connected by tight junctions with an extremely high electrical resistivity of at least 0.1 Ω·m. The blood-brain barrier allows the passage of water, some gases, and lipid-soluble molecules by passive diffusion, as well as the selective transport of molecules such as glucose and amino acids that are crucial to neural function. On the other hand, the blood-brain barrier may prevent the entry of lipophilic, potential neurotoxins by way of an active transport mechanism mediated by P-glycoprotein. Astrocytes are necessary to create the blood-brain barrier. A small number of regions in the brain, including the circumventricular organs (CVOs), do not have a blood-brain barrier.

The blood-brain barrier occurs along all capillaries and consists of tight junctions around the capillaries that do not exist in normal circulation. Endothelial cells restrict the diffusion of microscopic objects (e.g., bacteria) and large or hydrophilic molecules into the cerebrospinal fluid (CSF), while allowing the diffusion of small or hydrophobic molecules (e.g., O₂, CO₂, and hormones). Cells of the barrier actively transport metabolic products such as glucose across the barrier with specific proteins. This barrier also includes a thick basement membrane and astrocytic endfeet.

This “barrier” results from the selectivity of the tight junctions between endothelial cells in CNS vessels that restricts the passage of solutes. At the interface between blood and the brain, endothelial cells are stitched together by these tight junctions, which are composed of smaller subunits, frequently biochemical dimers that are trans membrane proteins such as occluding, claudins, junctional adhesion molecule (JAM), or ESAM, for example. Each of these trans membrane proteins is anchored into the endothelial cells by another protein complex that includes zo-1 and associated proteins.

The blood-brain barrier is composed of high-density cells restricting passage of substances from the bloodstream much more than do the endothelial cells in capillaries elsewhere in the body. Astrocyte cell projections called astrocytic feet (also known as “glia limitans”) surround the endothelial cells of the BBB, providing biochemical support to those cells. The BBB is distinct from the quite similar blood-cerebrospinal fluid barrier that is a function of the choroidal cells of the choroid plexus, and from the blood-retinal barrier, which can be considered a part of the whole realm of such barriers.

Several areas of the human brain are not on the brain side of the BBB. Some examples of this include the circumventricular organs, the roof of the third and fourth ventricles, capillaries in the pineal gland on the roof of the diencephalon and the pineal gland. The pineal gland secretes the hormone melatonin “directly into the systemic circulation”, thus the blood-brain barrier, does not affect melatonin.

The blood-brain barrier acts very effectively to protect the brain from most pathogens. Thus, blood borne infections of the brain are very rare. Infections of the brain that do occur are often very serious and difficult to treat. Antibodies are too large to cross the blood-brain barrier, and only certain antibiotics are able to pass. In some cases, a drug has to be administered directly into the cerebrospinal fluid, (CSF), where it can enter the brain by crossing the blood-cerebrospinal fluid barrier. However, not all drugs that are delivered directly to the CSF can effectively penetrate the CSF barrier and enter the brain. The blood-brain barrier becomes more permeable during inflammation. This allows some antibiotics and phagocytes to move across the BBB. However, this also allows bacteria and viruses to infiltrate the BBB. Examples of pathogens that can traverse the BBB and the diseases they cause include Toxoplasma gondii which causes toxoplasmosis, spirochetes like Borrelia which causes Lyme disease Group B streptococci which causes meningitis in newborns, and Treponema pallidum which causes syphilis. Some of these harmful bacteria gain access by releasing cytotoxins like pneumolysin, which have a direct toxic effect on brain micro vascular endothelium and tight junctions.

There are also some biochemical poisons that are made up of large molecules that are too big to pass through the blood-brain barrier. This was especially important in more primitive times when people often ate contaminated food. Neurotoxins such as botulinum in the food might affect peripheral nerves, but the blood-brain barrier can often prevent such toxins from reaching the central nervous system, where they could cause serious or fatal damage.

The blood brain barrier (BBB) is formed by the brain capillary endothelium and excludes from the brain about 100% of large-molecule neurotherapeutics and more than 98% of all small-molecule drugs. Overcoming the difficulty of delivering therapeutic agents to specific regions of the brain presents a major challenge to treatment of most brain disorders. In its neuroprotective role, the blood-brain barrier functions to hinder the delivery of many potentially important diagnostic and therapeutic agents to the brain. Therapeutic molecules and antibodies that might otherwise be effective in diagnosis and therapy do not cross the BBB in adequate amounts.

Mechanisms for drug targeting in the brain involve going either “through” or “behind” the BBB. Modalities for drug delivery/Dosage form through the BBB entail its disruption by osmotic means; biochemically by the use of vasoactive substances such as bradykinin; or even by localized exposure to high-intensity focused ultrasound (HIFU). Other methods used to get through the BBB may entail the use of endogenous transport systems, including carrier-mediated transporters such as glucose and amino acid carriers; receptor-mediated transcytosis for insulin or transferrin; and the blocking of active efflux transporters such as p-glycoprotein. However, vectors targeting BBB transporters, such as the transferrin receptor, have been found to remain entrapped in brain endothelial cells of capillaries, instead of being ferried across the BBB into the cerebral parenchyma. Methods for drug delivery behind the BBB include intracerebral implantation (such as with needles) and convection-enhanced distribution. Mannitol can be used in bypassing the BBB.

### SUMMARY

The disclosure provides for a tubular human BBB in vitro device that can emulate a BBB and may be employed to observe the effect of drug targets on neurons, in particular astrocytes, that are in proximity with capillary endothelial cells, e.g., brain capillary endothelial cells, separated by a porous polymeric tubular membrane. Exposing the endothelial side of the device (vascular mimic) to therapeutic challenges and measuring the effects of the therapeutic drug candidates allows for the determination whether the drug candidates are transported across the BBB (neuroendothelial cells as a barrier to the astrocytes). In one embodiment, the system is a closed loop system. In one embodiment, a medium, e.g., tissue culture media, blood or any physiologically compatible solution, may be introduced to the endothelial cells, optionally under physiologic pressure. The neurons are cultured in the same or a different solution than the endothelial cells. In one embodiment, the pressure is varied over time. In one embodiment, one or more compounds are introduced to the “luminal” side of the device (endothelial cells), and the effect on the neurons is detected, e.g., whether the one or more compounds are cytotoxic or exert an inhibitory or excitatory effect.

In one embodiment, a viable and functional human tubular BBB mimic device is provided having a multilayer human cell stack may be formed of, among other materials, a porous polymeric tubular membrane; extracellular matrix or a component thereof, other biomolecules, or a synthetic polymer; astrocytes; extracellular matrix or a component thereof, other biomolecules, or a synthetic polymer; and brain capillary endothelial cells.

In one embodiment, a viable and functional human tubular BBB mimic device is provided having a multilayer human cell stack may be formed of a porous polymeric tubular membrane; extracellular matrix or a component thereof; astrocytes; extracellular matrix or a component thereof; and brain capillary endothelial cells, which device can simulate blood or drug infused fluid circulating speeds and pressures found in the human body. The device is thus useful in, among other things, assessing pressure induced drug diffusion kinetics as well as efficacy in crossing the BBB.

In one embodiment, a viable and functional human tubular BBB mimic device is provided having a multilayer human cell stack may be formed of a porous polymeric tubular membrane; extracellular matrix or a component thereof; astrocytes; extracellular matrix or a component thereof; and brain capillary endothelial cells, which device is employed to measure the effects of therapeutic drug compounds that cross the tubular BBB into the astrocytes by techniques such as chemical and/or optical measurement techniques.

These and other objects and advantages will become apparent from the following detailed description.

### BRIEF DESCRIPTION OF DRAWINGS

FIG. 1 shows an end perspective cross sectional view of one embodiment of the device.

FIG. 2 shows an end perspective cross sectional view of small molecules crossing the BBB into the neurons and exterior cell media.

FIG. 3 shows a side view cross section of one embodiment with blood flow.

### DETAILED DESCRIPTION

The following discussion is directed towards the various embodiments. Although one or more of these embodiments may be preferred, the embodiments disclosed should not be interpreted, or otherwise used, as a limiting the scope of the disclosure, including the claims. In addition one skilled in the art will understand that the following description has broad application, and the discussion of any embodiment is meant only to be exemplary of that embodiment, and not intended to intimate that the scope of the disclosure, including the claims, is limited to that embodiment.

As discussed above, there are challenges in having an in vitro model that can emulate a vascular BBB for the screening of potential therapeutic compounds such as small molecules and biomolecules for various brain related diseases.

In one embodiment, a tubular in vitro blood brain barrier device is provided. The device includes a tubular chamber having an interior surface; a first layer comprising a plurality of mammalian neurons disposed on a second layer having one or more agents that are biocompatible and are adhered to at least some of the plurality of neurons, wherein the first layer is separated from the interior surface of the tubular chamber by a volume; a third layer comprising a tubular hollow fiber having pores, an interior surface and an exterior surface, which exterior surface of the third layer is disposed on the second layer; and a fourth layer having a plurality of endothelial cells disposed on a fifth layer having one or more agents that are biocompatible and are adhered to at least some of the plurality of endothelial cells, which fifth layer is disposed on the interior surface of the hollow fiber, wherein the fourth layer forms a tube having a lumen having an annular volume. In one embodiment, the second layer comprises gelatin, collagen, hyaluronic acid, cellulose, chemically modified cellulose, silicone, chitosan, vegetable protein, agar, polyacrylamide, polyvinylalcohol, polyols, fibronectin, vitronectin, laminin, matrigel, polylysine, polyvinylpyrrolidone, or other polypeptides, or any combination thereof. In one embodiment, the fifth layer comprises gelatin, collagen, hyaluronic acid, cellulose, chemically modified cellulose, silicone, chitosan, vegetable protein, agar, polyacrylamide, polyvinylalcohol, polyols, fibronectin, vitronectin, laminin, matrigel, polylysine, polyvinylprylidone, or other polypeptides, or any combination thereof. In one embodiment, the hollow fiber comprises polysulphone, polyvinylidene fluoride, fluoropolymers, polyethylene, polypropylene, nylon, polyester, cellulose, cellulose acetate, cellulose nitrate, polyacrylonitrile, polylactide, or polycaprolactone, or any combination thereof. In one embodiment, a wall of the hollow fiber has a thickness from about 1 to 50 microns. In one embodiment, a wall of the hollow fiber has a thickness from about 5 to 10 microns. In one embodiment, an inner diameter of the hollow fiber ranges from 10 microns to 1 millimeter. In one embodiment, an inner diameter of the hollow fiber ranges from 50 to 150 microns. In one embodiment, the pores of the hollow fiber have a molecular weight cutoff of 100 to 50,000 KDa. In one embodiment, the pore size of the hollow fiber size allows for passage of molecules of less than 5000 KDa but not greater than 5000 KDa. In one embodiment, the mammalian neurons are astrocytes, e.g., human astrocytes. In one embodiment, the endothelial cells comprise human brain capillary endothelial cells. In one embodiment, the thickness of the second layer or the fifth layer is from about 10 nanometers to 250 microns. In one embodiment, the fourth layer comprises a single layer of endothelial cells.

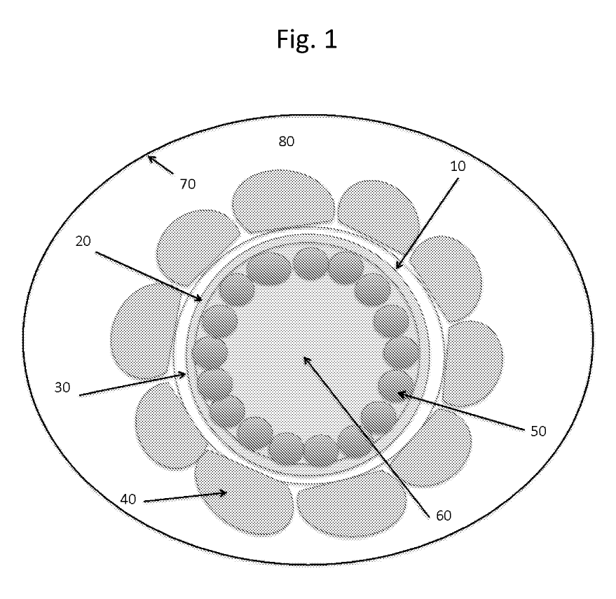

Referring to FIG. 1, in one embodiment a method of fabricating a human tubular blood brain barrier mimic is provided, which mimic has a multilayer cross section profile having an exterior chamber 70, that confines nutrient fluids 80 on the neuron side of the BBB, astrocytes 40 attached to extracellular matrix (ECM) 30, that is astrocyte compatible coated onto a hollow micro polymeric fiber 10 that is partially porous to small molecules and biomolecules, an interior confined by the hollow fiber comprising ECM 20 that is compatible with neuro endothelial cells coated onto the interior of the microfiber, e.g., human neuro capillary endothelial cells 50 attached to the ECM 20, and nutrient fluid 60. The nutrient fluids on both the interior and exterior of the hollow fiber may be different to nourish and support the cell growth and viability present on either side of the polymeric hollow fiber wall. The nutrient fluids are circulated through the system to both nourish and sweep metabolic waste away from the living cells. The circulating fluid may also be pressurized and pulsed at 1 Hz or higher to simulate blood flow. The circulating fluid may also carry drug candidates and be added to the interior of the hollow fibers only, to observe if they pass through the endothelial barrier to the exterior of the chamber and or into the neurons, e.g., human astrocytes.

Referring to FIG. 1, in one embodiment, a micro hollow fiber 10 is provided that is or can be fabricated from a number of polymeric materials such as polysulphone, polyvinylidene fluoride, fluoropolymers, polyethylene, polypropylene, nylon, polyester, cellulose, cellulose acetate, cellulose nitrate, polyacrylonitrile, polylactide, polycaprolactone, or any combination of the aforementioned polymers. The hollow fiber 10 walls can vary in thickness from about 1 to 50 microns, e.g., a thickness of about 5 to 10 microns. The hollow fiber inner diameter may range from 10 microns to 1 millimeter, e.g., 50 to 150 microns. In addition the hollow fiber may be porous. E.g., so as to allow small molecules from molecular weights of 100 to 50,000 KDa to pass through the porous fiber wall 10. In one embodiment, the pore size is less than 5000 KDa as most molecules that can pass through the human BBB are less than that molecular weight.

FIG. 1, 20 shows the next layer in the BBB stack. 20 is an ECM or ECM-like material (formed of ECM or a component thereof, or a synthetic polymer) which coats the interior of the hollow fiber in order to act as an anchor layer for the attachment of the neuron capillary endothelial cells 50. The ECM material is generally water-soluble and when applied contains from 0.01 to 10% ECM in solution. In one embodiment, the ECM is generally around from 0.1 to 1% and is pumped through the interior of the hollow fiber 10 until there is full coverage of the hollow fiber wall interior. The ECM or a component thereof, or a synthetic polymer, may be formed of one or more of the following materials, including but not limited gelatin, collagen, hyaluronic acid, cellulose, chemically modified cellulose, silicone, chitosan, vegetable protein, agar, polyacrylamide, polyvinylalcohol, polyols, fibronectin, vitronectin, laminin, matrigel, polylysine, polyvinylprylidone, or other polypeptides, or any combination of the aforementioned materials, with or without crosslinking. The ECM or ECM-like material may also contain adsorbed or absorbed polypeptides such as RED, REDV or KREDVY to further enhance cell adhesion to the ECM or ECM-like material. In one embodiment gelatin is used as the ECM 20.

In one embodiment, the cells used for the brain capillary endothelial cell (BCEC) 50 layer include cells from the hCMEC/D3 BBB cell line, which was derived from human temporal lobe microvessels; were immortalized with hTERT and SV40 large T antigen; and have been extensively characterized for brain endothelial phenotype and are a model of human blood-brain barrier (BBB) function. The cell line may be purchased from EMD Millipore Corporation in Temecula, Calif. This BCEC layer 50 may be used to study pathological and drug transport mechanisms with relevance to the central nervous system. The cells may be loaded into the hollow fibers via pumping and generally between 5 and 500,000,000 million cells are loaded into a hollow fiber device with multiple hollow fibers. The cells are loaded and allowed to expand to cover the entire hollow fiber wall and form tight junctions between the cells. During the cell loading the hollow fiber device may be rotated 360 degrees for over about a 24-hour period to assist in uniform cell converge and distribution on the hollow fiber walls 10.

FIG. 1, 30 shows the next layer in the BBB stack. 30 is an ECM or ECM-like material (formed of ECM or a component thereof, or a synthetic polymer, respectively) which coats the exterior of the hollow fibers in order to act as an anchor layer for the attachment on the neuron astrocytes cells 40. The ECM or ECM-like material is generally water-soluble and contains from 0.01 to 10% ECM in solution when applied. In one embodiment the ECM or ECM-like material is generally around from 0.1 to 1% and is pumped through the exterior of the hollow fiber 10 until there is full coverage of the cell wall exterior. The ECM or ECM-like material can be formed of any material, including but not limited to gelatin, collagen, hyaluronic acid, cellulose, chemically modified cellulose, silicone, chitosan, vegetable protein, agar, polyacrylamide, polyvinylalcohol, polyols, fibronectin, vitronectin, laminin, matrigel, polylysine, polyvinylprylidone, or other polypeptides, or any combination of the aforementioned materials, with or without crosslinking. The ECM or ECM-like material may also contain adsorbed or absorbed polypeptides such as; RED, REDV and KREDVY to further enhance neuron 40 cell adhesion to the ECM. In one embodiment, hyaluronic acid is used as the ECM 30. It should be noted that the interior (IF) 60 and exterior (EF) 80 of the hollow fiber 10 are physically separated such that different solutions can be added to the either side independently. After the EF side of the fibers is coated with ECM the astrocytes 40 may be added using the same process utilized for the BCEC 50 cells.

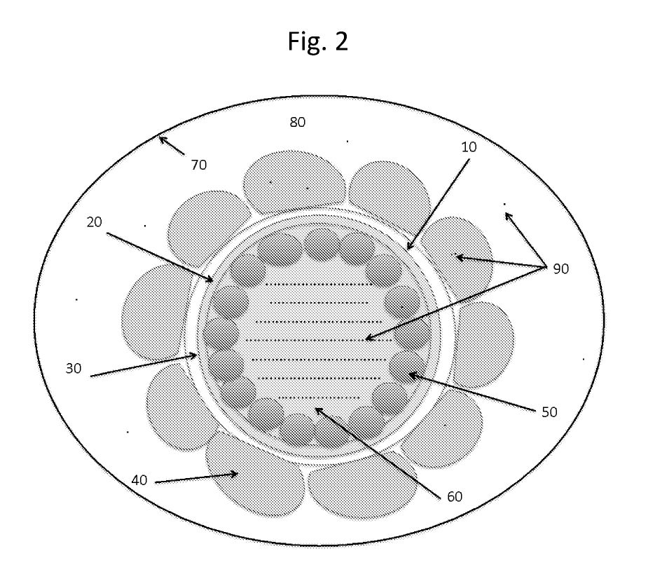

The hollow fiber device may then be allowed to age from 7 to 60 days to allow the cells to mature and form tight junction connections to each other and form a solid layer of cells on the both interior and exterior of the hollow fibers 10. During this time the neuro capillary cells are nourished, e.g., with EndoGro™ which is available from EMD Millipore of Concord Mass., and the astrocytes are also nourished, e.g., by BrainPhys™ which is available from StemCell Technologies, Vancouver Canada. The cells and device are then incubated at a temperature of 37° C. in a 95/5% oxygen to carbon dioxide atmosphere. In one embodiment, the cells are matured. Then the interior of the device may be charged with a molecule of interest FIG. 2, 90 and allowed to equilibrate. If the molecules 90 pass the BCEC they diffuse outwards towards the astrocytes after passing through the porous polymer fiber 10, where they may be processed and/or diffuse into the culture media. At that time one can sample the media from a sample port and look for the molecule or molecule byproducts of interest by techniques well known in the art such as gas chromatography, gel electrophoresis, Mass Spectrometry or Fluorescence.

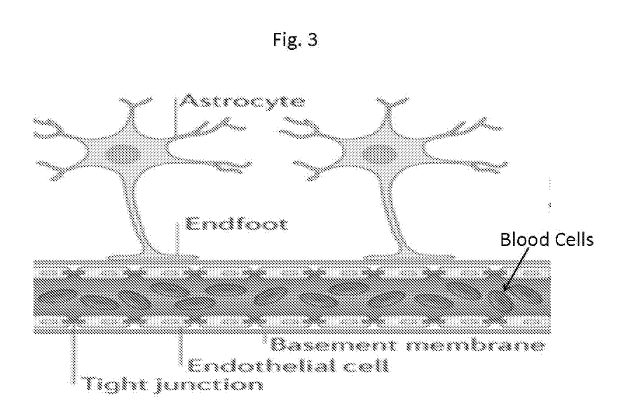

Referring to FIG. 3, this diagram shows a side view cross section of one embodiment in order to better understand how the device can be used for blood circulation. In FIG. 3 the basement membrane is the porous hollow fiber polymer tube 10. It depicts the endothelial cells 50 that have formed tight junctions due to maturing. The exterior of the basement membrane shows astrocytes 40 attached to the hollow fiber exterior surface. Blood cells or any liquid for that matter can be pumped into the interior of the device on the endothelial side of the hollow fiber to simulate blood flows and pressures that are experienced in the human body. Molecular diffusion of molecules may be dependent on actual pressures, temperatures and pulse rates of the liquids passing through the device.

The above discussion is meant to be illustrative of the principle and various embodiments. Numerous variations and modifications will become apparent to those skilled in the art once the above disclosure is fully appreciated. It is intended that the following claims be interpreted to embrace all such variations and modifications.

1. A tubular in vitro blood brain barrier device, comprising:
a tubular chamber having an interior surface;
a first layer comprising a plurality of mammalian neurons in contact with a second layer having one or more agents that are biocompatible and are optionally adhered to at least some of the plurality of neurons, wherein the first layer is separated from the interior surface of the tubular chamber by a volume;
a third layer comprising a tubular hollow fiber having pores, an interior surface and an exterior surface, which exterior surface of the third layer is in contact with the second layer; and
a fourth layer having a plurality of endothelial cells in contact with a fifth layer having one or more agents that are biocompatible and are optionally adhered to at least some of the plurality of endothelial cells, which fifth layer is in contact with the interior surface of the hollow fiber, wherein the fourth layer forms a tube having a lumen having an annular volume.

2. The device of claim I wherein the second layer comprises gelatin, collagen, hyaluronic acid, cellulose, chemically modified cellulose, silicone, chitosan, vegetable protein, agar, polyacrylamide, polyvin.ylalcohol, polyols, fibronectin, vitronectin, laminin, matrigel, polylysine, polyvinylprylidone, or other polypeptides, or any combination thereof; or wherein the fifth layer comprises gelatin. collagen, hyaluronic acid, cellulose, chemically modified cellulose, silicone, chitosan, vegetable protein, agar, polyacrylamide, polyvinylalcohol, polyols, fibronectin, vitronectin, laminin, matrigel, polylysine, polyvinylprylidone, or other polypeptides, or any combination thereof; or wherein the hollow fiber comprises polysulphone, polyvinylidene fluoride, fluoropolymers polyethylene, polypropylene, nylon, polyester, cellulose, cellulose acetate, cellulose nitrate, polyacrylonitrile, polylactide, or polycaprolactone, or any combination thereof.

3-4. (canceled)

5. The device of claim 1 wherein a wall of the hollow fiber has a thickness from about 1 to about 50 microns or has a thickness from about 5 to about 10 microns.

6. (canceled)

7. The device of claim 1 wherein an inner diameter of the hollow fiber ranges from about 10 microns to about 1 millimeter or wherein an inner diameter of the hollow fiber ranges from about 50 to about 150 microns.

8. (canceled)

9. The device of claim 1 wherein the pores of the hollow fiber have a molecular weight cutoff of about 100 to about 50,000 KDa or wherein the pore size of the hollow fiber size allows for passage of molecules of less than 5000 KDa.

10. (canceled)

11. The device of claim 1 wherein the mammalian neurons are astrocytes.

12. (canceled)

13. The device of claim 1 wherein the endothelial cells comprise capillary endothelial cells.

14. (canceled)

15. The device of claim 1 wherein the thickness of the second layer or the fifth layer is from about 10 nanometers to 250 microns.

16. The device of claim 1 wherein the fourth layer comprises a single layer of endothelial cells.

17. The device of claim 1 wherein the mammalian neurons are human astrocytes and the endothelial cells comprise human brain capillary endothelial cells.

18. The device of claim 1 wherein the second layer or the fifth layer comprises hyaluronic acid.

19. (canceled)

20. The device of claim 1 wherein the second layer or the fifth layer further comprises cell adhesion peptide or polypeptide comprising RED, REDV and KREDVY.

21-22. (canceled)

23. A method of using a barrier device, comprising:
providing the device of claim 1, wherein the volume that separates the first layer from the interior surface of the tubular chamber comprises a first aqueous liquid and the annular volume comprises a second aqueous liquid, wherein the second aqueous liquid comprises one or more test compounds; and
detecting whether the one or more tests compounds or a metabolite thereof are present in the first aqueous liquid or whether the one or more tests compounds alter the activity of the mammalian neurons.

24. The method of claim 23 wherein the one or more test compounds are introduced to the second aqueous liquid after the second aqueous liquid is introduced to the annular volume or wherein the one or more compounds alter the viability of the mammalian neurons or wherein the activity that is altered is action potential, impedance or conduction velocity.

25. The method of claim 23 wherein the first liquid and the second liquid before the one or more test compounds are provided to the second aqueous liquid, are different or wherein the second aqueous liquid is introduced under positive pressure which pressure is optionally physiological pressure.

26-29. (canceled)

30. A method of making a tubular in vitro blood brain barrier device, comprising:
providing a tubular chamber having an interior surface, the lumen of which comprises a tubular hollow fiber having pores, an interior surface and an exterior surface;
coating the exterior surface of the tubular hollow fiber with a second layer having one or more agents that are biocompatible and contacting the second layer with a plurality of neurons which adhere to the second layer, thereby forming a first layer, wherein the first layer is separated from the interior surface of the tubular chamber by a volume; and
coating the interior surface of the tubular hollow fiber with a fifth layer having one or more agents that are biocompatible and contacting the fifth layer with a plurality of endothelial cells which adhere to the fifth layer, thereby forming a fourth layer, wherein the fifth layer has an interior diameter which has an annular volume.

31. The method of claim 30 wherein the plurality of neurons that are contacted with the second layer are in a culture medium or wherein the plurality of endothelial cells that are contacted with the fifth layer are in culture medium.

32-33. (canceled)

34. The method of claim 30 wherein the plurality of endothelial cells are contacted with the fifth layer before or after the plurality of neurons are contacted with the second layer.

35. (canceled)

36. The method of claim 30 wherein the plurality of endothelial cells are contacted with the fifth layer when the plurality of neurons are contacted with the second layer.

37. The method of claim 30 further comprising introducing a first aqueous liquid to the annular volume; or introducing a second aqueous liquid to the volume separating the interior surface of the tubular chamber and the first layer; or introducing a first aqueous liquid to the annular volume and introducing a second aqueous liquid to the volume separating the interior surface of the tubular chamber and the first layer.

38-39. (canceled)
